(19) United States
(12) Patent Application Publication
(10) Pub. No.: US 2025/0009546 A1
(43) Pub. Date: Jan. 9, 2025

(54) PANNUS RETRACTOR-AN APRON FOR REMOVING EXCESS SKIN AND FAT HANGING FROM THE ABDOMEN

(71) Applicants: BOARD OF TRUSTEES OF THE UNIVERSITY OF ARKANSAS, Little Rock, AR (US); WASHINGTON REGIONAL MEDICAL CENTER, Fayetteville, AR (US)

(72) Inventors: Shivani Koundinya, Centerton, AR (US); Alexandra Johnston, Fayetteville, AR (US); Breanna Kilgore, El Dorado, AR (US); Claire Dildy, Fayetteville, AR (US); Johnna Winfrey, Siloam Springs, AR (US)

(73) Assignees: BOARD OF TRUSTEES OF THE UNIVERSITY OF ARKANSAS, Little Rock, AR (US); WASHINGTON REGIONAL MEDICAL CENTER, Fayetteville, AR (US)

(21) Appl. No.: 18/762,493

(22) Filed: Jul. 2, 2024

Related U.S. Application Data
(60) Provisional application No. 63/524,790, filed on Jul. 3, 2023.

(51) Int. Cl. A61F 5/03

(52) U.S. Cl. CPC A61F 5/03

(57) ABSTRACT

Embodiments of the present disclosure pertain to an abdominal stabilizer that is operational to wrap around an abdomen of a subject. The abdominal stabilizer includes a central region operational to be placed on the subject's abdomen, a proximal end operational to be immobilized on a first back region of the subject, a distal end on the opposite side of the proximal end that is operational to be immobilized on a second back region of the subject, an outer surface operational to face away from the subject, and an inner surface operational to face towards the subject. The abdominal stabilizers may also include a first strap and a second strap positioned above the central region and operational to adhere to a chest region of the subject. Further embodiments of the present disclosure pertain to methods of lifting an abdominal pannus of a subject through the utilization of the aforementioned abdominal stabilizers.

### CROSS-REFERENCE TO RELATED APPLICATIONS

This application claims priority to U.S. Provisional Patent Application No. 63/524,790, filed on Jul. 3, 2023. The entirety of the aforementioned application is incorporated herein by reference.

### BACKGROUND

A need exists for more effective systems and methods for pannus retraction during various procedures. Numerous embodiments of the present disclosure aim to address the aforementioned need.

### SUMMARY

In some embodiments, the present disclosure pertains to an abdominal stabilizer that is operational to wrap around an abdomen of a subject. In some embodiments, the abdominal stabilizer includes a central region operational to be placed on the subject's abdomen, a proximal end operational to be immobilized on a first back region of the subject, and a distal end on the opposite side of the proximal end that is operational to be immobilized on a second back region of the subject. The abdominal stabilizers of the present disclosure also include an outer surface operational to face away from the subject, and an inner surface operational to face towards the subject. In some embodiments, the inner surface includes an adhesive operational to adhere to the skin of the subject.

In some embodiments, the abdominal stabilizers of the present disclosure also include a first strap and a second strap positioned above the central region. In some embodiments, the first strap and the second strap are operational to adhere to a chest region of the subject and thereby provide additional stability. In some embodiments, the first strap and second strap are operational to form an X-shaped structure on the chest region of the subject.

Additional embodiments of the present disclosure pertain to a method of lifting an abdominal pannus of a subject through the utilization of the abdominal stabilizers of the present disclosure. In some embodiments, the methods of the present disclosure may include: placing the central region of the abdominal stabilizer on the subject's abdomen such that the outer surface faces away from the subject and the inner surface faces towards the subject; immobilizing the proximal end of the abdominal stabilizer on a first back region of the subject; and immobilizing the distal end of the abdominal stabilizer on the second back region of the subject, where the first back region and the second back region are on opposite sides of one another. As a result, the immobilizing of the proximal end and the distal end facilitates the lifting of the abdominal pannus.

### BRIEF DESCRIPTION OF THE DRAWINGS

A better understanding of the present invention can be obtained when the following detailed description is considered in conjunction with the following drawings, in which:

FIG. 1 shows a top view of an abdominal stabilizer of the present disclosure.

FIGS. 2A-2C show the application of an abdominal stabilizer of the present disclosure to a subject.

FIGS. 3A-3C shows the use of an abdominal stabilizer of the present disclosure in pannus retraction.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The panniculus is an apron of excess skin and fat hanging from the abdomen below the waistline. Pannus retraction through various retention and retraction systems or techniques is utilized during procedures that require visualization or access to the abdominal or suprapubic region.

An example of a regularly adopted procedure is Cesarean section, also known as C-section or cesarean delivery. A Cesarean delivery is a procedure used to deliver a baby through a surgical incision made in the abdomen and uterus. Prior to the procedure, the patient will receive a subarachnoid block, a spinal anesthetic. Abdominal and uterine incisions are made to allow delivery of the baby through this route. Often, the baby needs to be guided through the incision route. Nurses or scrub technicians push on the upper part of the uterus, located above the naval, in order to direct the baby to the incision site. Once the baby is delivered, they are placed on the mother's chest for skin-to-skin contact. Such skin-to-skin contact is important as it helps reduce the baby's stress, as well as increase nutrient absorption from breast-feeding.

Due to the sometimes-emergent nature of a cesarean section or staffing constraints, the pannus retractors in their current state are impractical, as they cannot be easily placed by a lone staff member. Cumbersome and labor-intensive application methods can also draw attention to the application of the device, leading to additional embarrassment for the patient. Current devices on the market require multiple staff members for application.

Current C-section devices also increase the risk of skin folding and skin lesions, which further discomforts the patient during an invasive and vulnerable procedure. In addition, current devices sometimes hinder the necessary skin-to-skin contact needed between mothers and children and can cause deep embarrassment for patients who feel exposed and vulnerable during these types of procedures.

The World Health Organization reports that currently 1 in 5 births are delivered via Cesarean, with those rates expected to rise to nearly a third by 2023. With the rise in cesarean births, there is also a rise in obesity rates in the nation. For instance, the March of Dimes and peer-reviewed literature cite maternal obesity or increased body mass index (BMI) as a risk factor for Cesarean section. In fact, research points to a pre-pregnancy BMI weight greater than 30 as an independent risk factor for emergency Cesarean section. As obesity rises, so does the need for proper pannus retraction for cesarean deliveries.

In sum, a need exists for more effective systems and methods for pannus retraction during various procedures. For instance, managing the abdominal pannus of obese patients during cesarean deliveries is a challenge for surgeons and nurses. Current devices require more time and people, making them undesirable and embarrassing for the patient. Numerous embodiments of the present disclosure aim to address the aforementioned need.

### Abdominal Stabilizers

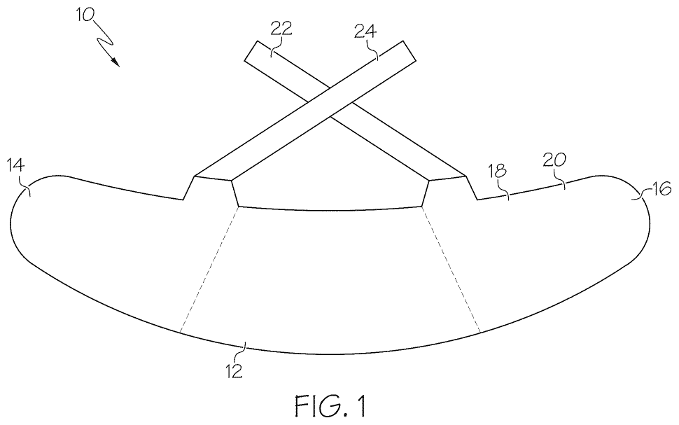

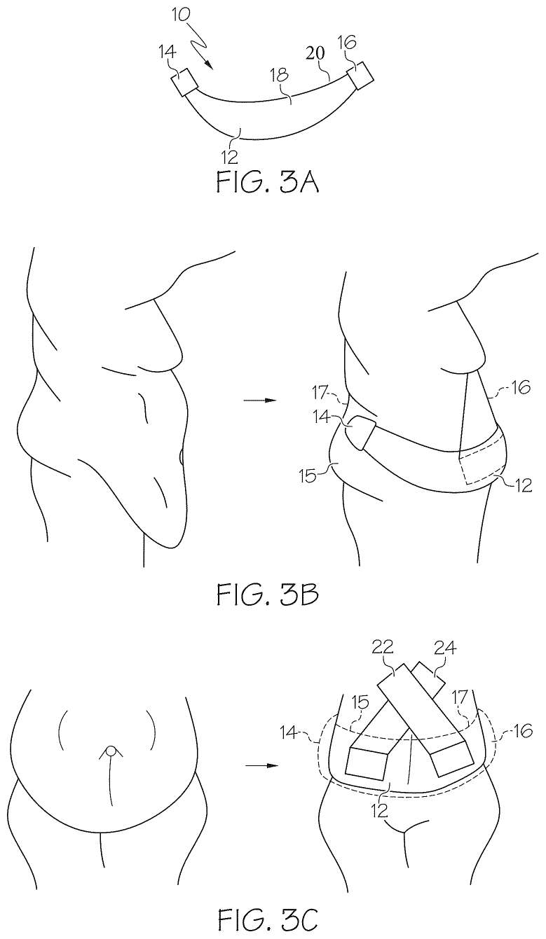

In some embodiments, the present disclosure pertains to an abdominal stabilizer. The abdominal stabilizers of the present disclosure may be illustrated as abdominal stabilizer 10 in FIGS. 1, 2A-2C, and 3A-3C.

As illustrated in FIGS. 1, 2A-2C, and 3A-3C, abdominal stabilizer 10 is operational to wrap around an abdomen of a subject. Abdominal stabilizer 10 includes a central region 12 that is operational to be placed on the subject's abdomen. Abdominal stabilizer 10 also includes a proximal end 14 operational to be immobilized on a first back region 15 of the subject, and a distal end 16 on the opposite side of the proximal end 14 that is operational to be immobilized on a second back region 17 of the subject. The first back region 15 and the second back region 17 are on the opposite sides of one another. In some embodiments, the width of central region 12 is higher than the width of each of proximal end 14 and distal end 16.

Abdominal stabilizer 10 also includes an outer surface 18 operational to face away from the subject, and an inner surface 20 operational to face towards the subject. In some embodiments, inner surface 20 includes an adhesive operational to adhere to the skin of the subject. In some embodiments, the adhesive includes a peelable cover. In some embodiments, the adhesive is at or near proximal end 14 of the inner surface. In some embodiments, the adhesive is at or near distal end 16 of the inner surface. In some embodiments, the adhesive is at or near central region 12 of the inner surface. In some embodiments, the adhesive spans the entire inner surface 20.

In some embodiments, inner surface 20 has a peelable backing that is perforated. In some embodiments, the peelable backing can be readily removed for exposure of an adhesive associated with inner surface 20. In some embodiments, the adhesive can be worn for up to 24 hours, thereby allowing an incision site to remain exposed.

In some embodiments, abdominal stabilizer 10 also includes a first strap 22 and a second strap 24 positioned above central region 12. In some embodiments, first strap 22 and second strap 24 are operational to adhere to a chest region of the subject and thereby provide additional stability. In some embodiments, the inner surfaces of first strap 22 and second strap 24 include an adhesive. In some embodiments, first strap 22 and second strap 24 are operational to form an X-shaped structure on the chest region of the subject.

The abdominal stabilizers of the present disclosure can have various structures and arrangements. For instance, in some embodiments, proximal end 14 and distal end 16 do not contact one another when wrapped around the abdomen of a subject. In some embodiments, the abdominal stabilizers of the present disclosure are operational to remain below the shoulders of the subject when wrapped around the abdomen of the subject. In some embodiments, the abdominal stabilizers of the present disclosure are operational to not extend beyond the shoulders of the subject when wrapped around the abdomen of the subject. In some embodiments, the abdominal stabilizers of the present disclosure lack shoulder straps.

The abdominal stabilizers of the present disclosure can have various shapes. For instance, in some embodiments, the abdominal stabilizers of the present disclosure are in the form of a curved structure. In some embodiments, central region 12 represents a base area of the curved structure. In some embodiments, proximal end 14 and distal end 16 represent outer edges of the curved structure. In some embodiments, the curved structure is in the form of a U-shaped structure.

The abdominal stabilizers of the present disclosure can have various compositions. For instance, in some embodiments, the abdominal stabilizers of the present disclosure can include medical grade plastics and adhesives. In some embodiments, the abdominal stabilizers of the present disclosure can include latex-free components to avoid any latex-related allergic reactions. In some embodiments, the abdominal stabilizers of the present disclosure can include antimicrobial components to prevent any postoperative infections.

The abdominal stabilizers of the present disclosure can have various uses. For instance, in some embodiments, the abdominal stabilizers of the present disclosure are operational to lift the abdominal pannus of the subject.

### Methods of Lifting an Abdominal Pannus

Additional embodiments of the present disclosure pertain to a method of lifting an abdominal pannus of a subject through the utilization of the abdominal stabilizers of the present disclosure. With reference to abdominal stabilizer 10 in FIGS. 1, 2A-2C, and 3A-3C for illustrative purposes, the methods of the present disclosure may include: placing the central region 12 of the abdominal stabilizer 10 on the subject's abdomen such that the outer surface 18 faces away from the subject and the inner surface 20 faces towards the subject; immobilizing the proximal end 14 of the abdominal stabilizer on a first back region 15 of the subject; and immobilizing the distal end 16 of the abdominal stabilizer on the second back region 17 of the subject, where the first back region 15 and the second back region 17 are on opposite sides of one another. As a result, the immobilizing of the proximal end 14 and the distal end 16 facilitates the lifting of the abdominal pannus. In some embodiments, the abdominal pannus is lifted away from a desired incision site.

In some embodiments where an abdominal stabilizer includes first strap 22 and second strap 24, the methods of the present disclosure also include a step of adhering first strap 22 and the second strap 24 of the abdominal stabilizer to a chest region of a subject. In some embodiments, the adhering includes forming an X-shaped structure of strap 22 and second strap 24 on the chest region of the subject.

In some embodiments, inner surface 20 of an abdominal stabilizer includes an adhesive operational to adhere to the skin of a subject. In some embodiments, the adhesive is at or near proximal end 14 of inner surface 20. In some of such embodiments, the immobilizing of proximal end 14 of the abdominal stabilizer on the first back region 15 of the subject includes using the adhesive to adhere the proximal end 14 to the first back region 15.

In some embodiments, the adhesive is at or near distal end 16 of inner surface 20. In some of such embodiments, the immobilizing of distal end 16 of the abdominal stabilizer on the second back region 17 of the subject includes using the adhesive to adhere the distal end 16 to the second back region 17.

In some embodiments, the adhesive is at or near central region 12 of inner surface 20. In some of such embodiments, placement of central region 12 of the abdominal stabilizer on the subject's abdomen includes using the adhesive to adhere central region 12 on the subject's abdomen.

The abdominal stabilizers of the present disclosure can be placed on a subject in various manners. For instance, in some embodiments, proximal end 14 and distal end 16 do not contact one another when wrapped around the abdomen of the subject. In some embodiments, the abdominal stabilizer remains below the shoulders of the subject when wrapped around the abdomen of the subject. In some embodiments, the abdominal stabilizer does not extend beyond the shoulders of the subject when wrapped around the abdomen of the subject.

### Applications and Advantages

The abdominal stabilizers and methods of the present disclosure can have numerous advantages. For instance, in some embodiments, a curved (e.g., U-shape) design of an abdominal stabilizer of the present disclosure prevents extra skin folds forming during the application of pannular lifting. In some embodiments, adhesives on the inner surfaces of the abdominal stabilizers of the present disclosure provide durable, 24-hour adhesion, which can allow for convenient and durable application of abdominal stabilizers during and after surgery. In some embodiments, X-fold straps above the abdominal stabilizers of the present disclosure allow for even force distribution across an attached abdominal stabilizer.

In some embodiments, the effective designs of the abdominal stabilizers of the present disclosure allow the stabilizer to be applied to a subject in a facile manner by a single user (e.g., a single nurse). For instance, in some embodiments, the effective designs of the abdominal stabilizers of the present disclosure allow stable pannus retention, thereby reducing application time and manpower required, while further empowering the patient, and improving the quality of patient care.

In some embodiments, the compact designs of the abdominal stabilizers of the present disclosure provide a competitive advantage over existing devices as it does not slip or interfere with drapes while users (e.g., nurses and scrub techs) are pushing on the abdomen. In some embodiments, the compact designs of the abdominal stabilizers of the present disclosure do not require an extra extender to fit all patients due to straps being present for extra support. As such, in some embodiments, the compact designs of the abdominal stabilizers of the present disclosure are more reliable, discreet, and simple to use than current pannus retraction solutions on the market.

As such, the abdominal stabilizers and methods of the present disclosure may be utilized for various purposes. For instance, in some embodiments, the abdominal stabilizers and methods of the present disclosure may be utilized to facilitate a medical procedure. In some embodiments, the methods of the present disclosure may also include a step of implementing the medical procedure. In some embodiments, the medical procedure includes, without limitation, Cesarean section surgery, vaginal delivery, femoral artery surgeries, heart catheter placement, hip replacements, abdominal surgeries, bariatric surgeries, weight loss surgeries, gynecological surgeries, pap smears, spinal surgeries, arterial scope procedures, urological surgeries, vasectomy, cervical procedures, cosmetic surgeries, gallbladder removal, trauma operations, breast reduction, rotator cuff surgery, intravenous stent placement, heart surgery, ultrasounds, kidney stone removal, neck-related medical procedures, imaging, wound care, or combinations thereof. In some embodiments, the medical procedure includes Cesarean section surgery.

In some embodiments, the abdominal stabilizers and methods of the present disclosure may be utilized for bariatric surgeries, such as gastric bypass, sleeve gastrectomy, and/or weight loss surgeries. For instance, in some embodiments, the abdominal stabilizers and methods of the present disclosure may be used to lift and secure excess abdominal tissue in patients undergoing bariatric surgeries, thereby providing better access and visibility for a surgeon.

In some embodiments, the abdominal stabilizers and methods of the present disclosure may be utilized for abdominal surgeries, such as hernia repairs, appendectomies, and/or bowel resections. In some embodiments, the abdominal stabilizers and methods of the present disclosure may be utilized for gynecological surgeries, such as hysterectomies and/or ovarian cyst removals. In some embodiments, the abdominal stabilizers and methods of the present disclosure may be utilized for urological surgeries, such as nephrectomies and/or prostatectomies.

In some embodiments, the abdominal stabilizers and methods of the present disclosure may be utilized for imaging, such as ultrasounds, CT scans and/or magnetic resonance imaging (MRI). In some embodiments, the lifting of the pannus by the abdominal stabilizers of the present disclosure can help provide more accurate imaging of underlying organs and structures.

In some embodiments, the abdominal stabilizers and methods of the present disclosure may be utilized for wound care, such as dressing changes. For instance, for patients with significant pannus, the abdominal stabilizers and methods of the present disclosure could be used to securely lift and hold excess tissue in place while healthcare professionals clean and dress wounds, ulcers, or incisions located beneath the pannus. This would make the wound care process more comfortable for the patient, while also allowing for more effective and efficient wound management.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Use of a Pannus Retractor in Cesarean Section

With reference again to FIGS. 1, 2A-2C and 3A-3C, this Example illustrates the use of an abdominal stabilizer 10 during a Cesarean section, which requires retracting the pannus up and away from the incision site.

As illustrated in FIG. 1, abdominal stabilizer 10 uses adhesives on inner surface 20 across the abdomen to lift the pannus away for the surgical sight. Abdominal stabilizer 10 spans around 35 inches across the abdomen and wraps around the back of the patient.

First strap 22 and second strap 24 across the front of abdominal stabilizer 10 are 6 inches each and can be placed in the ‘X’ formation for extra support for larger patients. While central region 12 is only 7.5 inches wide, it covers the entire lower half of the stomach without covering the chest. Straps 22 and 24 can be placed right under the breast area, giving access to the chest for skin-to-skin contact and allowing a smooth surface when nurses push on the abdomen during delivery. Additionally, proximal end 14 and distal end 16 are approximately 6 inches wide.

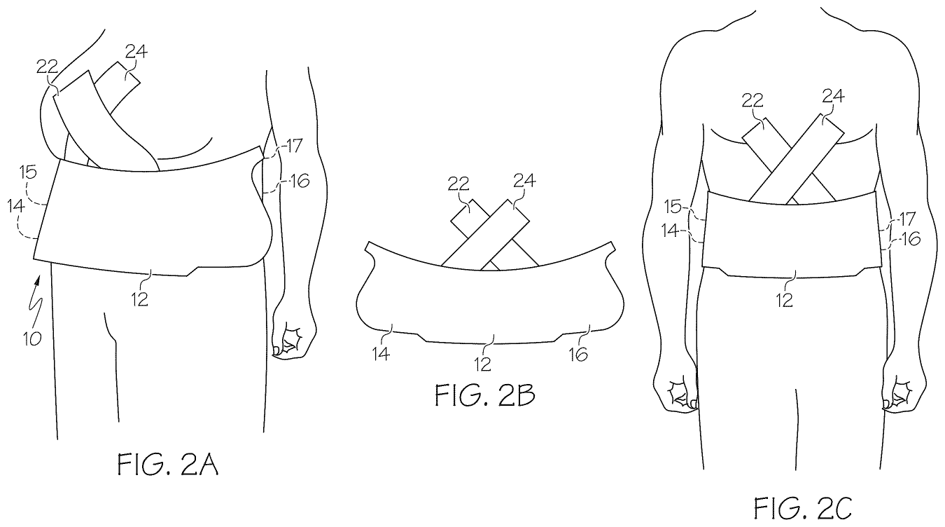

FIGS. 2A-2C provide a mockup of abdominal stabilizer 10 using a paper cut out and silk tape to act as first strap 22 and second strap 24 for a prototype. In actuality, the entire abdominal stabilizer 10 could be one adhesive material. Abdominal stabilizer 10 could mainly be in a U-shape design, allowing proximal end 14 and distal end 16 to tape to the back of the patient. Straps 22 and 24 could be placed under the breast region to hold up abdominal stabilizer 10, thereby not interfering with the draping placed on top. FIG. 2B displays abdominal stabilizer 10 laid flat on a hard surface.

Prior to the application of abdominal stabilizer 10 and to a planned Cesarean section, the patient will receive a subarachnoid block (spinal). The spinal is protected with a plastic sterile covering during the operation. Immediately following the administration of the spinal, abdominal stabilizer 10 will be attached to the left side of the patient's back while they are in a seated and upright position on the operating table. When laying the patient down, a nurse will use a block to lift or “bump” the woman's right hip for the operation. At this moment, the nurse has access to a portion of the patient's lower back and will attach the first end of the device.

Abdominal stabilizer 10 has a peelable inner surface 20, which can be pulled off to expose an adhesive on inner surface 20 as it is being applied. The nurse can complete the application of abdominal stabilizer 10 to the patient by reaching behind the patient and attaching adhesive-exposed inner surface 20 of proximal end 14 and distal end 16 to the back of the patient. As the patient is positioned to lay flat, the nurse can wrap and pull off the adhesive peel cover of inner surface 20.

Central region 12 of abdominal stabilizer 10 will then be adhered across the pannus region as the patient is lying flat on the table. The attached first strap 22 and second strap 24 can then be used to provide additional support of the pannus, if necessary.

To use the first strap 22 and second strap 24, inner surface 20 behind the straps is peeled off to expose the adhesive. Straps 22 and 24 are then placed in an X-pattern from the top of abdominal stabilizer 10, across the abdomen, and under the opposite breast of the patient. This design choice allows for skin-to-skin contact between the mother and child and removes the slippery surface when the nurse needs to push on the abdomen.

Once applied to the patient, abdominal stabilizer 10 can be easily prepped over for the procedure. Abdominal stabilizer 10 can maintain adhesive properties for up to 24 hours after the procedure.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. An abdominal stabilizer operational to wrap around an abdomen of a subject, wherein the abdominal stabilizer comprises:
a central region operational to be placed on the subject's abdomen;
a proximal end operational to be immobilized on a first back region of the subject;
a distal end on the opposite side of the proximal end, wherein the distal end is operational to be immobilized on a second back region of the subject, and wherein the first back region and the second back region are on the opposite sides of one another;
an outer surface operational to face away from the subject; and
an inner surface operational to face towards the subject.

2. The abdominal stabilizer of claim 1, wherein the width of the central region is higher than the width of each of the proximal end and distal end.

3. The abdominal stabilizer of claim 1, wherein the inner surface comprises an adhesive operational to adhere to the skin of the subject.

4. The abdominal stabilizer of claim 3, wherein the adhesive comprises a peelable cover.

5. The abdominal stabilizer of claim 3, wherein the adhesive is at or near the proximal end of the inner surface.

6. The abdominal stabilizer of claim 3, wherein the adhesive is at or near the distal end of the inner surface.

7. The abdominal stabilizer of claim 3, wherein the adhesive is at or near the central region of the inner surface.

8. The abdominal stabilizer of claim 3, wherein the adhesive spans the entire inner surface.

9. The abdominal stabilizer of claim 1, wherein the abdominal stabilizer further comprises a first strap and a second strap positioned above the central region, wherein the first strap and the second strap are operational to adhere to a chest region of the subject and thereby provide additional stability.

10. The abdominal stabilizer of claim 9, wherein the inner surfaces of the first strap and the second strap comprise an adhesive.

11. The abdominal stabilizer of claim 9, wherein the first strap and the second strap are operational to form an X-shaped structure on the chest region of the subject.

12. The abdominal stabilizer of claim 1, wherein the abdominal stabilizer is in the form of a curved structure, wherein the central region represents a base area of the curved structure, and wherein the proximal end and distal end represent outer edges of the curved structure.

13. The abdominal stabilizer of claim 12, wherein the curved structure is in the form of a U-shaped structure.

14. The abdominal stabilizer of claim 1, wherein the abdominal stabilizer is operational to lift the abdominal pannus of the subject.

15. A method lifting an abdominal pannus of a subject through the utilization of an abdominal stabilizer, wherein the abdominal stabilizer comprises:
a central region,
a proximal end,
a distal end on the opposite side of the proximal end,
an outer surface, and
an inner surface; and
wherein the method comprises:
placing the central region of the abdominal stabilizer on the subject's abdomen such that the outer surface faces away from the subject and the inner surface faces towards the subject;
immobilizing the proximal end of the abdominal stabilizer on a first back region of the subject; and
immobilizing the distal end of the abdominal stabilizer on the second back region of the subject,
wherein the first back region and the second back region are on opposite sides of one another, and
wherein the immobilizing of the proximal end and the distal end facilitates the lifting of the abdominal pannus.

16. The method of claim 15, wherein the abdominal stabilizer further comprises a first strap and a second strap positioned above the central region, wherein the method further comprises a step of adhering the first strap and the second strap of the abdominal stabilizer to a chest region of the subject.

17. The method of claim 16, wherein the adhering comprises forming an X-shaped structure of the first strap and the second strap on the chest region of the subject.

18. The method of claim 15, wherein the inner surface comprises an adhesive operational to adhere to the skin of the subject.

19. The method of claim 18, wherein the adhesive is at or near the proximal end of the inner surface, and wherein the immobilizing of the proximal end of the abdominal stabilizer on the first back region of the subject comprises using the adhesive to adhere the proximal end to the first back region.

20. The method of claim 18, wherein the adhesive is at or near the distal end of the inner surface, and wherein the immobilizing of the distal end of the abdominal stabilizer on the second back region of the subject comprises using the adhesive to adhere the distal end to the second back region.

21. The method of claim 18, wherein the adhesive is at or near the central region of the inner surface, and wherein placement of the central region of the abdominal stabilizer on the subject's abdomen comprises using the adhesive to adhere the central region on the subject's abdomen.

22. The method of claim 15, wherein the proximal end and the distal end do not contact one another when wrapped around the abdomen of the subject.

23. The method of claim 15, wherein the abdominal stabilizer does not extend beyond the shoulders of the subject when wrapped around the abdomen of the subject.

24. The method of claim 15, wherein the method is utilized to facilitate a medical procedure.

25. The method of claim 24, wherein the medical procedure is selected from the group consisting of Cesarean section surgery, vaginal delivery, femoral artery surgeries, heart catheter placement, hip replacements, abdominal surgeries, bariatric surgeries, weight loss surgeries, gynecological surgeries, pap smears, spinal surgeries, arterial scope procedures, urological surgeries, vasectomy, cervical procedures, cosmetic surgeries, gallbladder removal, trauma operations, breast reduction, rotator cuff surgery, intravenous stent placement, heart surgery, ultrasounds, kidney stone removal, neck-related medical procedures, imaging, wound care, or combinations thereof.

26. The method of claim 24, wherein the medical procedure comprises Cesarean section surgery.

27. The method of claim 24, further comprising a step of implementing the medical procedure.
